(19) United States
(12) Patent Application Publication
(10) Pub. No.: US 2012/0292517 A1
(43) Pub. Date: Nov. 22, 2012

(54) REAL-TIME IMAGING DOSIMETER SYSTEMS AND METHOD

(75) Inventor: Enrique Wilmar Izaguirre, St. Louis, MO (US)

(73) Assignee: WASHINGTON UNIVERSITY, St. Louis, MO (US)

(21) Appl. No.: 13/473,112

(22) Filed: May 16, 2012

Related U.S. Application Data
(60) Provisional application No. 61/487,917, filed on May 19, 2011.

(51) Int. Cl. A61N 5/10; G01T 1/20

(52) U.S. Cl. 250/362; 250/363.01; 250/367

(57) ABSTRACT

A radiation therapy system including a linear accelerator configured to emit a beam of radiation and a dosimeter configured to detect in real-time the beam of radiation emitted by the linear accelerator. The dosimeter includes at least one linear array of scintillating fibers configured to capture radiation from the beam at a plurality of independent angular orientations, and a detection system coupled to the at least one linear array, the detection system configured to detect the beam of radiation by measuring an output of the scintillating fibers.

### CROSS-REFERENCE TO RELATED APPLICATION

This application claims the benefit of U.S. Provisional Application No. 61/487,917 filed May 19, 2011, which is incorporated herein in its entirety.

### BACKGROUND

Radiation therapy (also referred to as “radiation oncology”) is used to treat a significant number of cancer patients. Radiation therapy involves the application of precise doses of radiation to a tumorous site, and involves complicated treatments utilizing linear accelerators (LINACs), such as intensity modulated radiation therapy (IMRT), volumetric modulated arc therapy (VMAT), stereotactic radiosurgery (SRS), and stereotactic body radiation therapy (SBRT). Ionizing radiation damages the DNA of the tumor, causing it to die, or reproduce more slowly. The rate of destruction of the tumor is dependent on the dose absorbed by the tumor tissue and the radiosensitiviy of the tumor cell to the ionizing radiation.

Radiation dosimetry involves determining the dose of radiation delivered to tissue and is a fundamental technique for performing quality assurance (QA) of instruments that deliver ionizing irradiation and for verifying the quality of the treatment received by the patient. Radiation dosimetry procedures may be utilized to measure or calculate the absorbed dose, to verify the dose of radiation and/or to calibrate the equipment providing the source of radiation. In at least some known radiation therapy systems, QA is done prior to treatment by measuring cumulative doses delivered to a phantom and comparing the measured doses delivered to the phantom with expected doses computed in a treatment planner. These QA procedures may be performed using single point ionization chambers and/or diodes, arrays of ionization chambers and/or diodes, radiochromic films, and/or radiographic films.

Notably, using these QA protocols, the actual dose delivered to the patient is unknown. That is, using at least some known radiation therapy systems, the actual spatial and temporal dose delivered to the patient is not verifiable. Accordingly, if the radiation therapy system and/or LINAC malfunction due to, for example, a misplaced multi-leaf collimator (MLC) leaf, an enhanced dynamic wedge failure, an inadvertent physical wedge on the machine, and/or patient motion, such malfunctions will remain undetected. This could result in a patient unknowingly receiving more than or less than the expected dose of radiation.

### BRIEF DESCRIPTION

In one aspect, a radiation therapy system is provided. The radiation therapy system includes a linear accelerator configured to emit a beam of radiation and a dosimeter configured to detect in real-time the beam of radiation emitted by the linear accelerator. The dosimeter includes at least one linear array of scintillating fibers configured to capture radiation from the beam at a plurality of independent angular orientations, and a detection system coupled to the at least one linear array, the detection system configured to detect the beam of radiation by measuring an output of the scintillating fibers.

In another aspect, a dosimeter assembly is provided. The dosimeter assembly includes at least one linear array of scintillating fibers configured to capture, at a plurality of independent angular orientations, radiation from a beam of radiation emitted from a radiation therapy apparatus, and a detection system coupled to the at least one linear array, the detection system configured to detect in real-time the beam of radiation by measuring an output of the scintillating fibers.

In yet another aspect, a method for real-time verification of a beam of radiation emitted from a radiation therapy apparatus is provided. The method includes providing at least one linear array of scintillating fibers positioned in a path of the beam of radiation, the at least one linear array configured to capture radiation from the beam at a plurality of independent angular orientations, acquiring a measured output of the scintillating fibers using a detection system including a processing device, and determining, using the processing device, a difference between the measured output of the scintillating fibers and an expected output of the scintillating fibers.

### BRIEF DESCRIPTION OF THE DRAWINGS

The embodiments described herein may be better understood by referring to the following description in conjunction with the accompanying drawings.

FIG. 1 is a schematic diagram of an exemplary external beam radiation therapy system.

FIG. 2 is a perspective view of an exemplary linear array dosimeter that may be used with the system of FIG. 1.

FIG. 3 is a perspective view of the dosimeter of FIG. 2.

FIG. 4 is a schematic diagram of an alternative array that may be used with the dosimeter of FIG. 2.

FIG. 5 is a schematic diagram of a dosimeter electronics assembly that may be used with the dosimeter of FIG. 2.

FIGS. 6A-6G are matrices and images demonstrating a beam reconstruction method.

FIG. 7 is a flowchart of an exemplary method for real-time verification of a beam of radiation emitted from a radiation therapy system.

### DETAILED DESCRIPTION

Embodiments provide a radiation therapy system including an in vivo real-time imaging dosimeter for external beam irradiation devices and patient specific quality assurance procedures. The dosimeter measures radiation doses delivered to a patient in real-time, improving patient safety and treatment accuracy in radiation oncology. The dosimeter utilizes a high-resolution two-dimensional scintillating fiber array to detect emitted radiation by the radiation system and the dose delivered to the patient according to the placement of the fiber array. By comparing a measured output of the linear array with an expected output, a processing device determines whether the radiation therapy system is operating within an acceptable threshold. By positioning the dosimeter between a linear accelerator (LINAC) and a patient, a beam of radiation emitted from the LINAC can be measured. Further, by positioning the dosimeter such that the patient is located between the LINAC and the dosimeter, the dose of radiation absorbed by the patient can be measured. Further, the dosimeter described in the embodiments herein has high detection efficiency, high signal to noise ratio, relatively inexpensive components, and compact sensing elements.

The methods and systems described herein may be used in various applications, including, but not limited to, external beam linear accelerators (LINACs), dynamic multi-leaf collimation (DMLC), static multi-leaf collimation (SMLC), intensity modulated radiation therapy (IMRT), volumetric modulated arc therapy (VMAT), arc beam therapy, stereotactic radiosurgery (SRS), stereotactic body radiation therapy (SBRT), and/or any mode of external beam therapy using photons and/or charged particles (e.g., electrons, protons, and carbon ions).

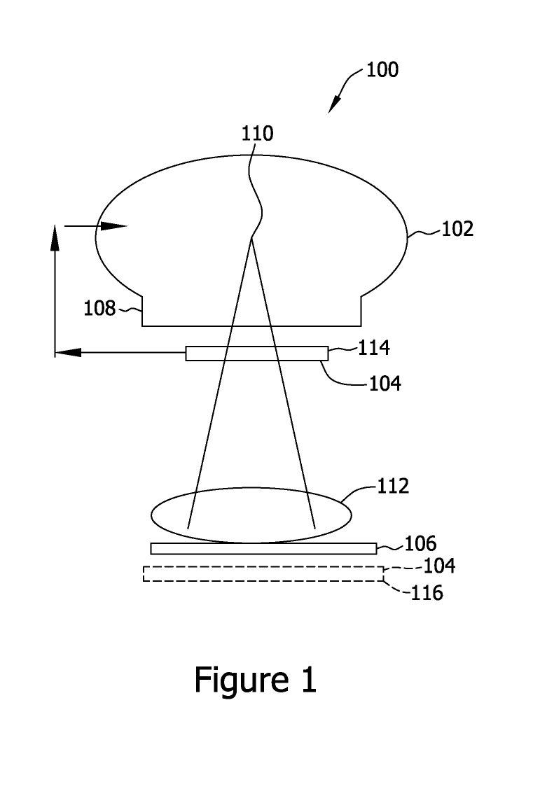

A schematic diagram of an exemplary external beam radiation therapy system 100 is shown in FIG. 1. System 100 includes a linear accelerator (LINAC) 102, a dosimeter 104, and a subject table 106. Dosimeter 104 is coupled to a head 108 of LINAC 102, such that a beam 110 of radiation emitted by LINAC 102 passes through dosimeter 104. Head 108 of LINAC 102 may also include a multi-leaf collimator (MLC) (not shown) for shaping beam 110. Further, dosimeter 104 is communicatively coupled to LINAC 102 such that beam 110 can be adjusted and/or corrected based on radiation detected by dosimeter 104, as described in detail below.

In operation of system 100, a patient 112 lies on subject table 106, such that beam 110 emitted by LINAC 102 strikes a desired treatment area of patient 112. Beam 110 may include photons, electrons, and/or protons emitted continuously or in periodic bursts. Alternatively, LINAC 102 may emit particles at any frequency that enables system 100 to function as described herein.

In some embodiments, dosimeter 104 is located between LINAC 102 and patient 112 at a first position 114. In first position 114, dosimeter 104 measures a fluence of beam 110 as beam is emitted from LINAC 102. Alternatively, dosimeter 104 is located below patient at a second position 116. In second position 116, dosimeter 104 may be mounted to subject table 106 and/or located below subject table 106. Further, as described in detail below, at least some components of dosimeter 104 may be plastic. Accordingly, in second position 116, dosimeter 104 may be embedded in subject table 106. In second position 116, dosimeter 104 operates in a transmission mode. That is, because patient 112 is located between LINAC 102 and dosimeter 104, dosimeter 104 measures a dose of radiation absorbed by patient 112 from beam 110, as described in detail below. In the embodiments described herein, dosimeter 104 may be located in first position 114 and/or second position 116.

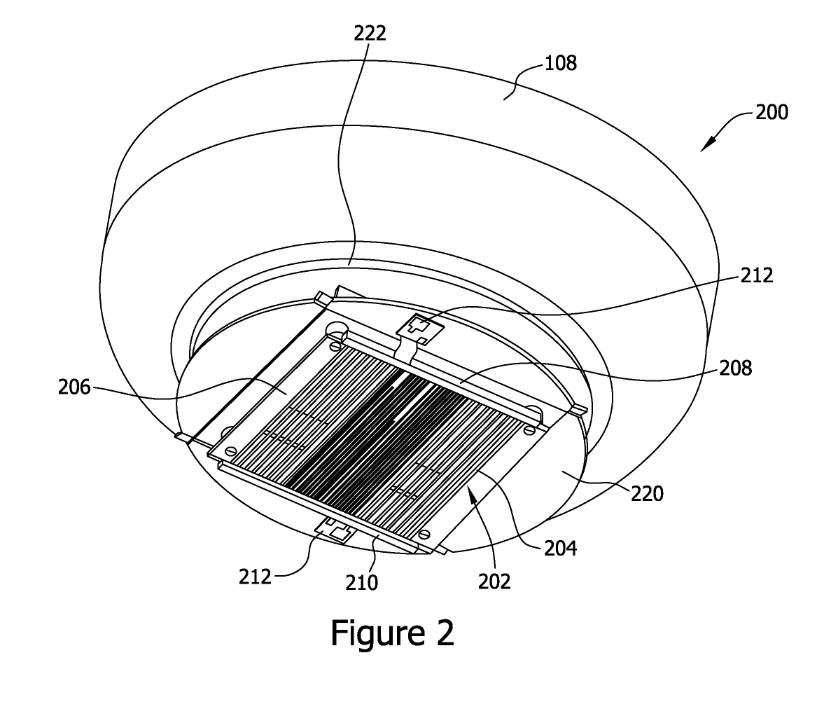

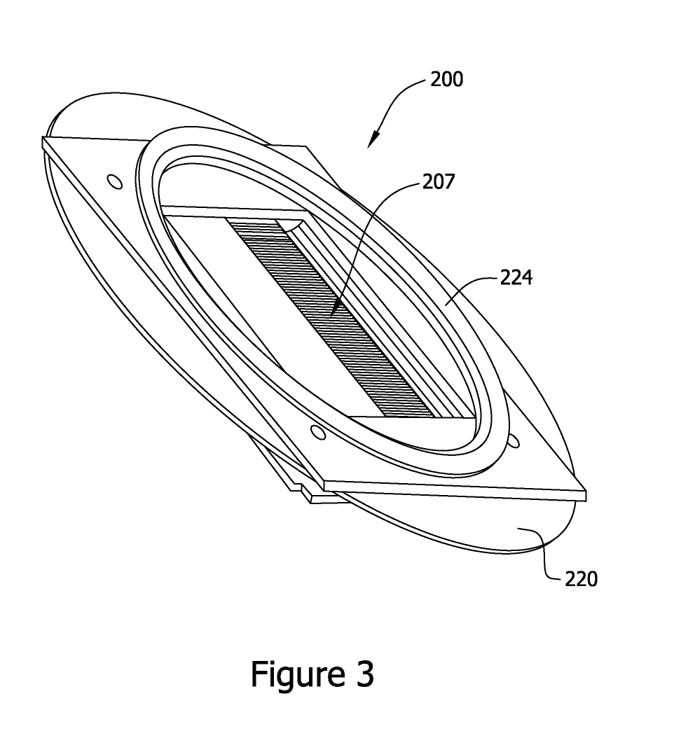

FIG. 2 is a perspective view of an exemplary linear array dosimeter 200 that may be used with the system of FIG. 1. FIG. 3 is a perspective view of dosimeter 200. Dosimeter 200 includes a linear array 202 of scintillating fibers 204 mounted to a fiber support frame 206. Linear array 202 may include any number of scintillating fibers 204 that enables dosimeter 200 to function as described herein. Dosimeter 200 may be installed on an already-existing LINAC head 108, or may be manufactured as part of LINAC head 108. Further, an aperture 207 is defined through dosimeter 200 such that beam 110 emitted from LINAC head 108 passes through linear array 202. In the embodiment shown in FIGS. 2 and 3, aperture 207 is substantially rectangular. Alternatively, aperture 207 may have any shape that enables dosimeter 200 to function as described herein.

In one embodiment, the number of scintillating fibers 204 is equal to a number of MLC leaf pairs (not shown) in LINAC head 108, and scintillating fibers 204 are spaced apart with separations that match a pitch of the MLC leaves, such that dosimeter 200 has a spatial resolution of approximately one millimeter, matching that of the MLC leaves. Alternatively, linear array 202 may include any number of scintillating fibers 204 at any separation that enables dosimeter 200 to function as described herein. Further, in embodiments with multiple linear arrays 202, described in detail below, at least one linear array 202 may be oriented along the MLC leaf pairs.

In the embodiment shown in FIGS. 2 and 3, each scintillating fiber 204 extends from a first end 208 of linear array 202 to a second end 210 of linear array 202. Further, first end 208 and second end 210 are coupled to detector electronics 212 for detecting radiation in beam 110 that passes through dosimeter 200. To detect radiation in beam 110, each fiber 204 generates a signal based on a line integral of the radiation transmitted through fiber 204, the line integral taken along the length of each fiber 204. Detector electronics 212 acquire this signal from each fiber 204. That is, dosimeter 200 uses detector electronics 212 to determine a total number of particles in beam 110 that strike each scintillating fiber 204 and/or a fluence of beam 110. Detector electronics 212 are described in detail below.

Scintillating fibers 204 are constructed using plastic substrates and are drawn using a fiber optic pulling machine. The composition of the scintillating fiber 204 is a clear plastic substrate doped with a scintillating organic or inorganic compound. The result is a material where the attenuation coefficient is similar to the substrate and therefore is water equivalent and thus tissue equivalent. Scintillating fibers 204 are longer than a maximum width of beam 110, such that first end 208 and second end 210 are located outside of beam 110. In one embodiment, each scintillating fiber 204 is a fiber or any other scintillating composition having a cross-sectional area of 0.52×0.52 square millimeters (mm) and a length greater than about 42 centimeters (cm). Alternatively, fibers may have a cross section of 1.5×1.5 square mm, 0.25×0.25 square mm, and/or any dimensions and/or shape that enable dosimeter 200 to function as described herein. In one embodiment, the cross-sectional dimension of each scintillating fiber 204 is selected based on a desired transmission and/or sensitivity of dosimeter 200. Scintillating fibers 204 may be constructed using polymethyl methacrylate (PMMA) or similar polymer materials which have an absorption coefficient similar to that of water and/or soft tissue.

In operation, LINAC 102 may emit, for example, a photon beam having an energy from about 2 to about 35 megaelectron volts (MeV), or an electron beam having an energy from about 2 to about 30 MeV. Scintillating fibers 204 have an output that is energy independent for energies above 0.5 MeV. Further, scintillating fibers 204 are stable, reproducible, and have a linear response proportional to a dose of incident radiation from beam 110. Moreover, scintillating fibers 204 are water impermeable and do not require a polarizing voltage for operation. Further, when exposed to beam 110, scintillating fibers 204 have a homogeneous photon and electron attenuation coefficient similar to soft tissue, minimal scattering, and a high beam transmission therethrough. While in one embodiment, scintillating fibers 204 form linear array 202, in some embodiments, linear array may include crystal rods, organic scintillators (e.g., plastics, acrylics), ionization chambers, and/or wire chambers.

In order to accurately detect radiation delivered to patient 112, scintillating fibers 204 have a water equivalent attenuation coefficient. Accordingly, linear array 202 accurately detects radiation in beam 110 that is delivered to patient 112. Further, fiber support frame 206, first end 208, and/or second end 210 may also be composed of water equivalent materials.

To detect beam 110 passing through dosimeter 200, data is collected with linear array 202 positioned at a number of angular orientations. That is, in the embodiment shown in FIGS. 2 and 3, dosimeter 200 includes a turntable 220 that is rotatably coupled to a LINAC accessory tray 222 of LINAC head 108. Turntable 220 is rotatably coupled to LINAC accessory tray 222 by a rotating ring 224. Alternatively, turntable may be rotatably coupled to LINAC head 108 using any suitable coupling means. Turntable 220 may also include a locking mechanism (not shown) for securing linear array 202 at different angular orientations. In operation, turntable 220 is rotated to acquire beam data with linear array 202 aligned with MLC leaves, at +30° with respect to MLC leaves, at +45° with respect to MLC leaves, at +90° with respect to MLC leaves, and at −45° with respect to MLC leaves. Alternatively, beam data may be acquired at any independent angular orientations and any number of orientations that enables dosimeter 200 to function as described herein. Further, beam data may be acquired using any number of linear arrays 202 and/or superpositions of linear arrays 202.

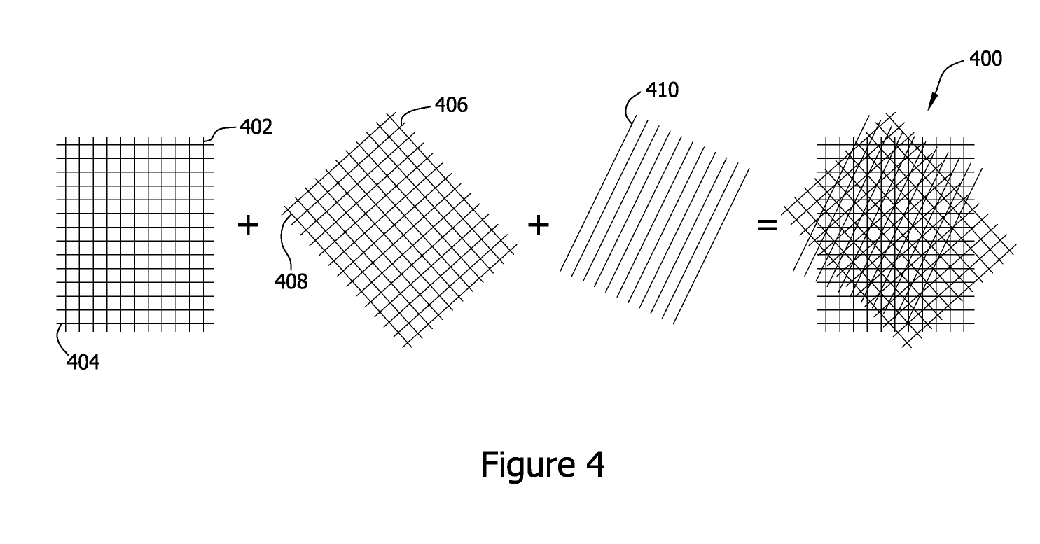

FIG. 4 is a schematic diagram of an alternative array 400 that may be used with dosimeter 200. Instead of a single linear array, such as linear array 202, array 400 includes five linear arrays superimposed and/or stacked upon one another. A first linear array 402 is aligned with MLC leaves in LINAC head 108. A second linear array 404 is oriented at +90° with respect to first linear array 402, a third linear array 406 is oriented at +45° with respect to first linear array 402, a fourth linear array 408 is oriented at −45° with respect to first linear array 402, and a fifth linear array 410 is oriented at +30° with respect to first linear array 402. Linear arrays 402, 404, 406, 408, and 410 are stacked on top of one another to form array 400. Using array 400, beam data may be acquired for five different angular orientations simultaneously, without rotating array 400. While five linear arrays are shown in FIG. 4, array 400 may include any number of linear arrays at any angular orientations that enable array 400 to function as described herein. In one embodiment, at least one linear array is oriented at an angular orientation that is independent from the angular orientation of another linear array. Using a single, rotated linear array (as shown in FIGS. 2 and 3) or multiple linear arrays (as shown in FIG. 4), a spatial and temporal distribution of the entire beam 110 can be acquired, as described in detail below. Accordingly, an accurate three-dimensional dose distribution of beam 110 can be calculated.

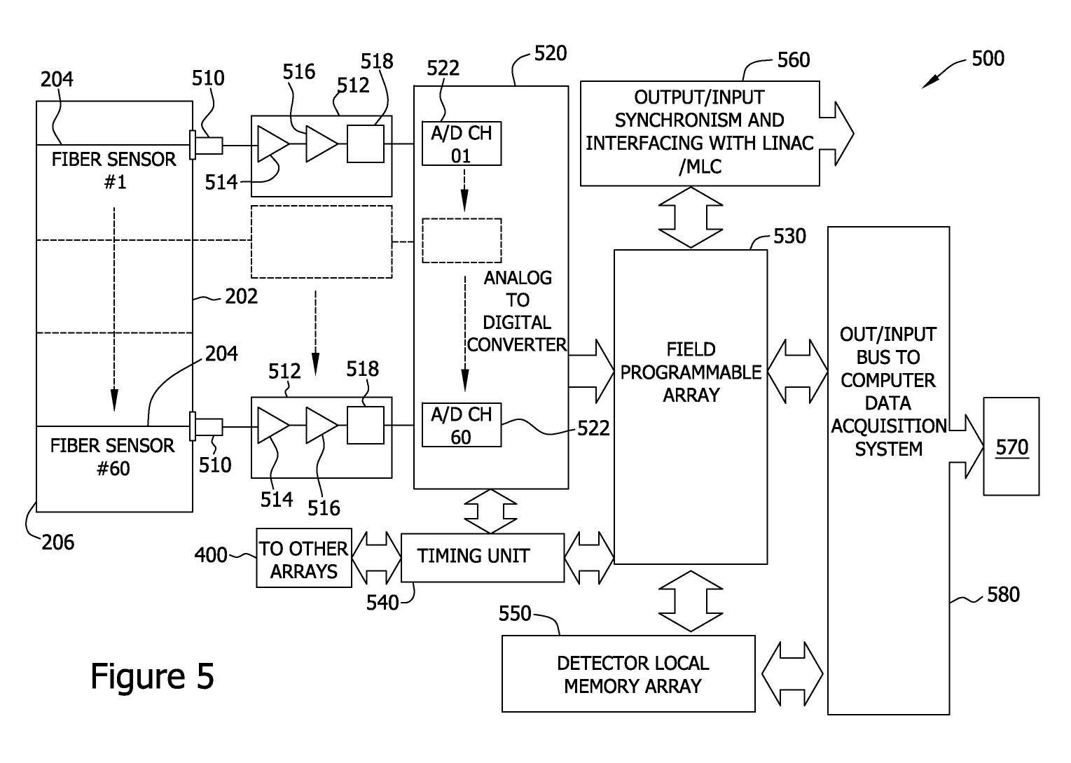

FIG. 5 is a schematic diagram of a dosimeter electronics assembly 500 that may be used with the dosimeter 200 of FIG. 2. Dosimeter electronics assembly 500 includes dosimeter 200, including linear array 202 of scintillating fibers 204 in fiber support frame 206. Dosimeter electronics assembly 500 forms a parallel data acquisition architecture to read the output of scintillating fibers 204 in array 202, as described in detail below. In one embodiment, dosimeter electronics assembly 500 measures an output of dosimeter 200 in approximately one hundred nanoseconds. As described above, scintillating fibers 204 and fiber support frame 206 are composed of water equivalent materials. For clarity, only two scintillating fibers 204 are illustrated in FIG. 5. However, dosimeter 200 may include any number of scintillating fibers 204 that enable dosimeter electronics assembly 500 to function as described herein.

Each scintillating fiber 204 is coupled to a photomultiplier 510. Photomultiplier 510 may be a plastic encapsulated solid state silicon photomultiplier, a photodiode, an avalanche photo-detector, and/or any device configured to detect an output of scintillating fiber 204, as described herein. When charged particles from beam 110 pass through scintillating fiber 204, photomultiplier 510 detects the light produced in scintillating fiber 204. The light produced in scintillating fiber 204 is proportional to the delivered dose of radiation. Thus, scintillating fibers 204 may be calibrated using known radiation doses to generate a table including the amount of light produced that corresponds to a given dosage.

By closely matching refraction indices between scintillating fiber 204 and photomultiplier 510, a high coupling efficiency between scintillating fiber 204 and photomultiplier 510 can achieved. Thus, dosimeter 200 has a relatively high signal to noise ratio. Photomultipliers 510 are located outside beam 110 emitted by LINAC head 108, and may be located at one or both ends of each scintillating fiber 204.

Each photomultiplier 510 is coupled to a signal conditioning circuit 512 that includes an amplifier 514, an adjustable threshold comparator 516, and an optical filter 518 for processing the output of photomultiplier 510. Amplifier 514 includes a matching impedance amplifier and an adjustable gain amplifier. Amplifier 514 can be adjusted to correct and/or balance sensitivity of a corresponding scintillating fiber 204. Further, signal conditioning circuit 512 may be used to initially calibrate each scintillating fiber 204. For example, over time, due to exposure of scintillating fibers 204 to beam 110, an output of scintillating fibers 204 may degrade and/or suffer an annealing process. These deficiencies can be compensated by periodically adjusting a gain of amplifier 514 to recalibrate scintillating fibers 204.

The output of scintillating fibers 204 may also include Cherenkov radiation. That is, scintillating fibers 204 emit green light due to incident particles from beam 110 and emit blue light due to Cherenkov radiation effects. Accordingly, optical filter 518 eliminates the output of scintillating fibers 204 due to Cherenkov radiation. In one embodiment, optical filter 518 is a plastic band pass filter and/or a high band pass filter.

Signal conditioning circuit 512 is coupled to an analog to digital convertor 520, such that the output signal of each photomultiplier 510, after being conditioned by signal conditioning circuit 512, is digitized. Analog to digital converter 520 uses one channel 522 for each scintillating fiber 204. Accordingly, the output signal for each scintillating fiber 204 may be digitized in less than ten microseconds. In the embodiment shown in FIG. 5, an output of the analog to digital converter 520 is coupled to a field programmable gate array (FPGA) 530. FPGA 530 is programmed to reconstruct beam data and process signals from analog to digital converter 520, as described in detail below. FPGA 530 may be programmed using, for example, a universal integrated circuit programmer Alternatively, analog to digital converter 520 may be coupled to any processing device capable of processing the signals and/or reconstructing beam data as described herein, such as an application-specific integrated circuit (ASIC).

In an embodiment where dosimeter 200 includes more than one linear array 202, such as linear arrays 402, 404, 406, 408, and 410 (shown in FIG. 4), the other arrays are coupled to analog to digital converter 520 and FPGA 530 through a timing unit 540.

FPGA 530 processes signals output from analog to digital converter 520 to generate measured data that includes the output of each scintillating fiber 204 and a corresponding time. Further, from the measured data, FPGA 530 can determine a spatial and temporal distribution of beam 110, an opening of beam 110, and accordingly, the position of MLC leaves in LINAC head 108, as described in detail below.

In the embodiment shown in FIG. 5, a memory array 550 is coupled to FPGA 530. Memory array 550 can include a computer-readable medium, such as, without limitation, random access memory (RAM), flash memory, a hard disk drive, a solid state drive, a diskette, a flash drive, a compact disc, a digital video disc, and/or any suitable memory that enables dosimeter electronics assembly 500 to function as described herein. In one embodiment, memory array 550 stores expectation data representing an expected output of dosimeter 200. More specifically, memory array 550 stores an expected output signal of each scintillating fiber 204. The expectation data may be calculated from a radiation treatment plan and/or based on previous operation of system 100 (shown in FIG. 1).

To determine whether system 100 is operating properly, FPGA 530 compares the output received from analog to digital converter 520 with the expectation data. That is, FPGA 530 compares an expected output signal of each scintillating fiber 204 with the measured output signal of each scintillating fiber 204. By calculating the difference between the expected and measured output, FPGA 530 calculates an error signal. If the error signal is greater than a predetermined threshold, FPGA 530 determines that the actual dose of radiation being administered to patient 112 by beam 110 is greater than or less than the expected dose of radiation, and that system 100 is malfunctioning. For example, FPGA 530 may be programmed to detect a malfunction when error signal is greater than 3% of the expected output. In one embodiment, the FPGA 530 calculates whether system 100 is malfunctioning in a time period shorter than a characteristic time for beam modulation and/or beam fluctuation of beam 110 passing through dosimeter 200. Accordingly, dosimeter electronics assembly 500 detects, in real-time, malfunctions in system 100. The comparison between the expected and measured output can be performed prior to treatment for QA purposes or during treatment for in vivo, real-time treatment verification and safety of patient 112. Further, the comparison can be performed continuously or periodically while system 100 is in operation.

In the event that FPGA 530 detects a malfunction in system 100, FPGA 530 may communicate and/or interface with LINAC 102 to adjust the MLC leaves in order to correct the malfunction. FPGA 530 communicates and/or interfaces with LINAC 102 through a feedback loop 560. For example, an output vector for each scintillating fiber 204 may be generated using FPGA 530. Using the generated vector, FPGA 530 may communicate with LINAC 102 to adjust MLC leaves in real-time, correcting treatment delivery. Accordingly, dosimeter electronics assembly 500 can detect and correct malfunctions of system 100 in real-time, minimizing any harm to patient 112. Further, FPGA 530, upon detecting a malfunction, may halt further treatment (e.g., stop LINAC 102 from emitting further radiation) and/or generate an alert to warn an operator of the malfunction. Moreover, the measured output of scintillating fibers 204 may be used to modify future radiation treatments of patient 112. For example, if patient 112 receives more radiation than expected, future dosages may be reduced. Similarly, if patient 112 receives less radiation than expected, future dosages may be increased.

In the embodiment shown in FIG. 5, for further processing, data analysis, and data recording, FPGA 530 is coupled to a data acquisition system 570 through a data bus 580. Memory array 550 is also coupled to data acquisition system 570. In one embodiment, data is transmitted to data acquisition system 570 periodically, for example, once every microsecond.

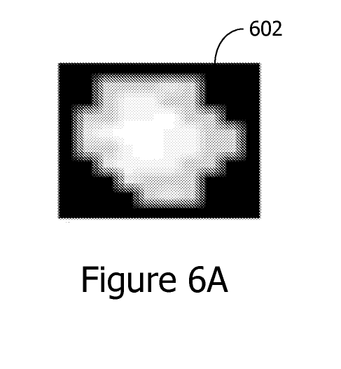

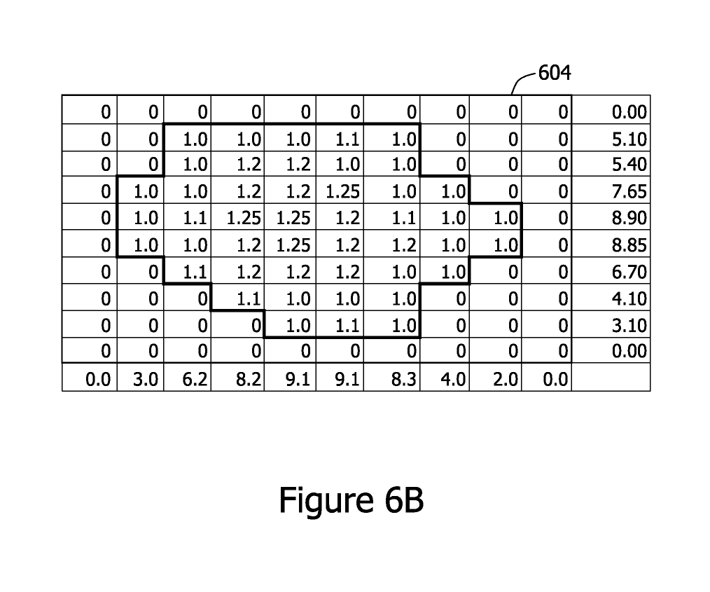

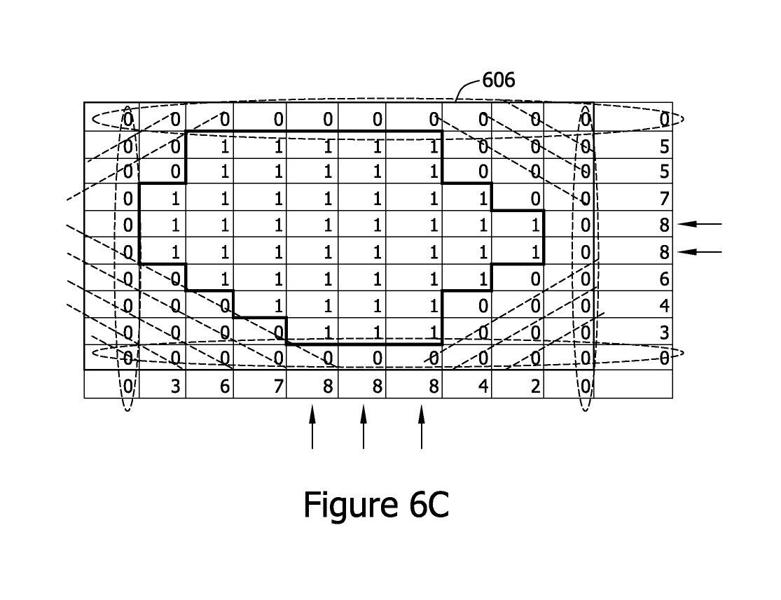

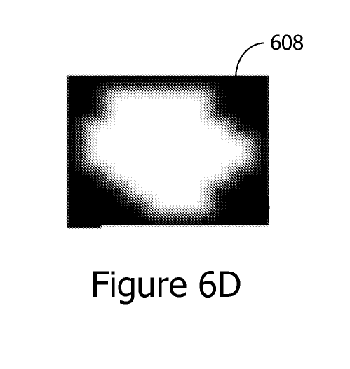

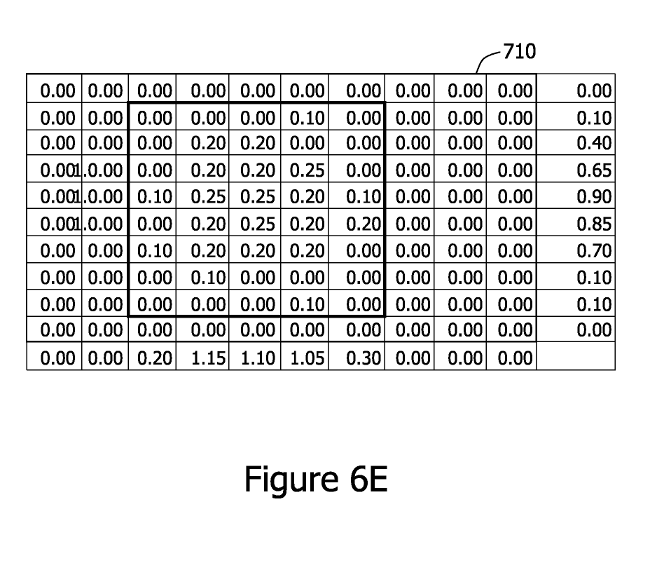

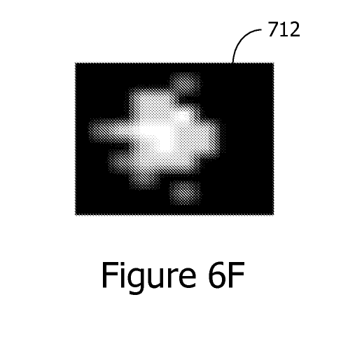

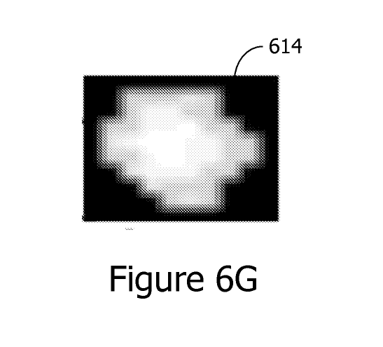

To determine a malfunction in system 100, as described above, FPGA 530 compares the measured output for each scintillating fiber 204 with the expected output for each scintillating fiber 204. Accordingly, to detect malfunctions, beam 110 need not be reconstructed. However, in some embodiments, FPGA 530 and/or data acquisition system 570 may reconstruct beam 110 using tomosynthesis and/or tomographic imaging methods. In one embodiment, FPGA 530 reconstructs beam 110, as shown and described in FIGS. 6A-6G.

FIG. 6A shows an image 602 of a beam to be reconstructed and FIG. 6B shows a matrix 604 of simplified fiber output data to demonstrate operation of an exemplary reconstruction algorithm. In matrix 604, for clarity, output data is shown for only ten vertical fibers 204 and ten horizontal fibers 204. However, output data for any number of fibers 204 may be processed to reconstruct the beam.

The algorithm normalizes the output data of scintillating fibers 204. FIG. 6C shows a matrix 606 with normalized output data. Scintillating fibers 204 that have relatively low and/or no output indicate the absence of beam 110. In FIG. 6C, the dotted lines represent orientations of scintillating fibers 204 with relatively low and/or no output. Using the normalized output data for each scintillating fiber 204, it can be determined which pixels in matrix 606 are located in the beam, and which pixels in matrix 606 are located outside the beam. This process may be carried out by beginning with fibers 204 that have a maximum or minimum normalized output, filling corresponding pixels with a normalized beam value (e.g., “1” for pixels in beam 110, and “0” for pixels outside beam 110), and subtracting the filled pixel values from the normalized fiber output. This process is repeated until the normalized fiber output for every scintillating fiber 204 is zero, and all pixels in matrix 606 are either identified as being in beam 110 or outside beam 110. FIG. 6D shows an image 608 of the reconstructed normalized beam. From matrix 606 and image 608, a spatial distribution and/or opening of beam 110 and positions of the MLC leaves can be determined.

For a more detailed reconstruction of beam 110, a difference signal representing the difference in pixel values between the raw beam data (shown in FIG. 6B) and the normalized beam data (shown in FIG. 6C) is calculated.

FIG. 6E shows a matrix 610 that includes the difference signal data, and FIG. 6F shows an image 612 of the difference signal. To reconstruct the detailed beam, the difference signal data is combined with the normalized beam data to iteratively correct the normalized beam data. FIG. 6G is an image 614 of the reconstructed detailed beam. The above-described reconstruction processes can be performed quickly, enabling real-time detection by dosimeter 200. In one embodiment, beam 110 is reconstructed in about five milliseconds.

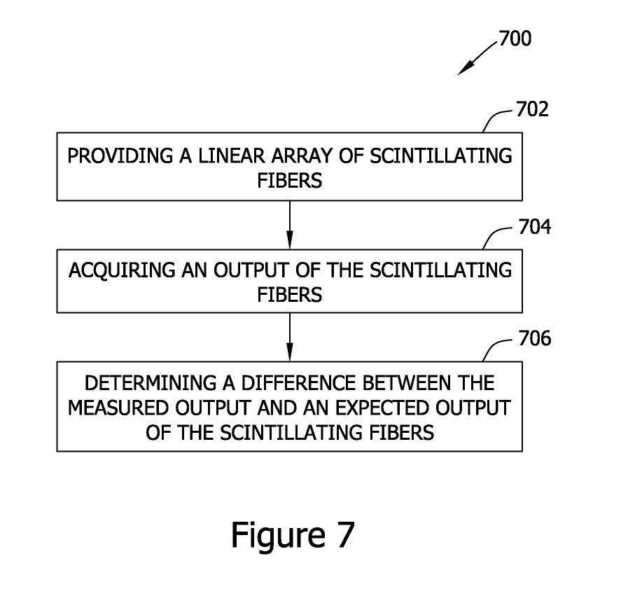

FIG. 7 is a flowchart of an exemplary method 700 for real-time verification of a beam of radiation emitted from a radiation therapy system, for example, beam 110 emitted from radiation therapy system 100. Method 700 includes providing 702 at least one linear array of scintillating fibers positioned in a path of the beam of radiation, such as linear array 202 and scintillating fibers 204. In one embodiment, a plurality of linear arrays are provided, such as in alternative array 400. A human operator may position the linear array prior to radiation treatment, or the linear array may already be located on the radiation therapy system. During operation of the radiation therapy system, an output of the scintillating fibers is acquired 704 and/or measured using a detection system and/or a processing device, such as dosimeter electronics assembly 500 and FPGA 530. The processing device determines 706 a difference between the measured output of scintillating fibers and an expected output of the scintillating fibers. If the determined difference suggests a malfunction of the radiation therapy system, the processing device may instruct the radiation therapy system to adjust the beam, halt treatment, and/or warn a human operator, as described in the above embodiments.

The embodiments described above provide a radiation therapy system including an in vivo real-time imaging dosimeter for quality assurance procedures. The dosimeter measures radiation doses delivered to a patient in real-time, improving patient safety and treatment accuracy in radiation oncology. The dosimeter utilizes a linear array of scintillating fibers to detect emitted radiation. By comparing a measured output of the linear array with an expected output, a processing device determines whether the radiation therapy system is operating within an acceptable threshold. By positioning the dosimeter between a LINAC and a patient, a beam of radiation emitted from the LINAC can be measured. Further, by positioning the dosimeter such that the patient is located between the LINAC and the dosimeter, the dose of radiation absorbed by the patient can be measured.

Additionally, the dosimeter described in the embodiments herein has high detection efficiency, high signal to noise ratio, and compact sensing elements. Moreover, the components used in the embodiments described herein reduce high costs associated with known radiation therapy systems. The dosimeter is not limited to the specific applications described herein, but may be utilized for a variety of techniques, for example pre-treatment dose delivery QA, LINAC commissioning, QA for patient treatment, QA of instruments that deliver radiation, and/or MLC calibration and QA.

It will be understood by those of skill in the art that information and signals may be represented using any of a variety of different technologies and techniques (e.g., data, instructions, commands, information, signals, bits, symbols, and chips may be represented by voltages, currents, electromagnetic waves, magnetic fields or particles, optical fields or particles, or any combination thereof). Likewise, the various embodiments described herein may be implemented as electronic hardware, computer software, or combinations of both, depending on the application and functionality. Moreover, the methods and systems described herein may be implemented or performed with a general purpose processor (e.g., microprocessor, conventional processor, controller, microcontroller, state machine or combination of computing devices), a digital signal processor (“DSP”), an application specific integrated circuit (“ASIC”), a field programmable gate array (“FPGA”) or other programmable logic device, discrete gate or transistor logic, discrete hardware components, or any combination thereof designed to perform the functions described herein. Similarly, steps of a method or process described herein may be embodied directly in hardware, in a software module executed by a processor, or in a combination of the two. A software module may reside in RAM memory, flash memory, ROM memory, EPROM memory, EEPROM memory, registers, hard disk, a removable disk, a CD-ROM, or any other form of storage medium known in the art. Although preferred embodiments of the present disclosure have been described in detail, it will be understood by those skilled in the art that various modifications can be made therein without departing from the spirit and scope of the disclosure as set forth in the appended claims.

A processing device, such as described herein, includes at least one or more processors or processing units and a system memory. The processing device typically also includes at least some form of computer readable media. By way of example and not limitation, computer readable media may include computer storage media and communication media. Computer storage media may include volatile and nonvolatile, removable and non-removable media implemented in any method or technology that enables storage of information, such as computer readable instructions, data structures, program modules, or other data. Communication media typically embody computer readable instructions, data structures, program modules, or other data in a modulated data signal such as a carrier wave or other transport mechanism and include any information delivery media. Combinations of any of the above are also included within the scope of computer readable media.

The order of execution or performance of the operations in the embodiments of the disclosure illustrated and described herein is not essential, unless otherwise specified. That is, the operations may be performed in any order, unless otherwise specified, and embodiments of the disclosure may include additional or fewer operations than those disclosed herein. For example, it is contemplated that executing or performing a particular operation before, contemporaneously with, or after another operation is within the scope of aspects of the disclosure.

When introducing elements of aspects of the disclosure or embodiments thereof, the articles “a,” “an,” “the,” and “said” are intended to mean that there are one or more of the elements. The terms “comprising,” including,” and “having” are intended to be inclusive and mean that there may be additional elements other than the listed elements.

This written description uses examples to disclose the disclosure, including the best mode, and also to enable any person skilled in the art to practice the disclosure, including making and using any devices or systems and performing any incorporated methods. The patentable scope of the disclosure is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

What is claimed is:

1. A radiation therapy system comprising:
a linear accelerator configured to emit a beam of radiation; and
a dosimeter configured to detect in real-time the beam of radiation emitted by said linear accelerator, said dosimeter comprising:
at least one linear array of scintillating fibers configured to capture radiation from the beam at a plurality of independent angular orientations; and
a detection system coupled to said at least one linear array, said detection system configured to detect the beam of radiation by measuring an output of the scintillating fibers.

2. A radiation therapy system in accordance with claim 1, wherein said detection system is configured to measure an output of the scintillating fibers by calculating a line integral of radiation transmitted through each fiber, the line integral calculated along the length of each fiber.

3. A radiation therapy system in accordance with claim 1, wherein said at least one linear array is rotatably coupled to said linear accelerator, such that said at least one linear array is adjustable to be positioned at the plurality of independent angular orientations.

4. A radiation therapy system in accordance with claim 1, wherein said at least one linear array comprises a plurality of linear arrays, wherein at least one of said plurality of linear arrays is oriented at a different angular orientation than another of said plurality of linear arrays.

5. A radiation therapy system in accordance with claim 1, further comprising a feedback loop communicatively coupling said dosimeter to said linear accelerator, such that the beam of radiation emitted by said linear accelerator may be adjusted based on the measured output of the scintillating fibers.

6. A radiation therapy system in accordance with claim 1, wherein said dosimeter is configured to detect in real-time a beam of radiation delivered to a patient.

7. A dosimeter assembly comprising:
at least one linear array of scintillating fibers configured to capture, at a plurality of independent angular orientations, radiation from a beam of radiation emitted from a radiation therapy apparatus; and
a detection system coupled to said at least one linear array, said detection system configured to detect in real-time the beam of radiation by measuring an output of the scintillating fibers.

8. A dosimeter assembly in accordance with claim 7, wherein said detection system comprises at least one photomultiplier coupled to a corresponding scintillating fiber, said at least one photomultiplier configured to detect light emitted from the corresponding scintillating fiber in response to the beam of radiation.

9. A dosimeter assembly in accordance with claim 7, wherein said detection system comprises at least one signal conditioning circuit coupled to a corresponding scintillating fiber, said at least one signal conditioning circuit comprising:
an amplifier configured to adjust a gain of the scintillating fiber output; and
a filter configured to remove a component of the scintillating fiber output caused by Cherenkov radiation.

10. A dosimeter assembly in accordance with claim 7, wherein said detection system comprises a processing device coupled to said at least one linear array, said processing device configured to determine a difference between the measured output of the scintillating fibers and an expected output of the scintillating fibers.

11. A dosimeter assembly in accordance with claim 10, further comprising a memory array coupled to said processing device and configured to store the expected output of the scintillating fibers.

12. A dosimeter assembly in accordance with claim 10, wherein when the difference between the measured output and the expected output is above a predetermined threshold, said processing device is configured to at least one of instruct the radiation therapy apparatus to stop emitting the beam of radiation, instruct the radiation therapy apparatus to adjust the beam of radiation, and alert an operator.

13. A dosimeter assembly in accordance with claim 10, wherein said processing device is configured to reconstruct the beam of radiation based on a maximum scintillating fiber output and a minimum scintillating fiber output.

14. A dosimeter assembly in accordance with claim 7 further comprising a support frame coupled to said at least one linear array, said support frame composed of a water equivalent material.

15. A dosimeter assembly in accordance with claim 7, wherein said at least one linear array comprises a plurality of linear arrays, each of the plurality of linear arrays oriented at one of the plurality of independent angular orientations.

16. A method for real-time verification of a beam of radiation emitted from a radiation therapy apparatus, said method comprising:
providing at least one linear array of scintillating fibers positioned in a path of the beam of radiation, the at least one linear array configured to capture radiation from the beam at a plurality of independent angular orientations; acquiring a measured output of the scintillating fibers using a detection system, the detection system including a processing device; and
determining, using the processing device, a difference between the measured output of the scintillating fibers and an expected output of the scintillating fibers.

17. A method in accordance with claim 16, further comprising reconstructing, using the processing device, the beam of radiation based on a maximum scintillating fiber output and a minimum scintillating fiber output.

18. A method in accordance with claim 16, further comprising
determining that the difference between the measured output and the expected output is above a predetermined threshold; and
instructing the radiation therapy apparatus to adjust the beam of radiation based on the determined difference.

19. A method in accordance with claim 16, further comprising
determining that the difference between the measured output and the expected output is above a predetermined threshold; and
alerting an operator that the difference is above the predetermined threshold.

20. A method in accordance with claim 16, further comprising:
storing the expected output of the scintillating fibers in a memory array coupled to the processing device.
